(19) United States
(12) Patent Application Publication
(10) Pub. No.: US 2004/0224014 A1
(43) Pub. Date: Nov. 11, 2004

(54) Non-hygroscopic pharmaceutical compositions containing non-hydrated quinoline carboxylic acids

(75) Inventors: Adnan Badwan, Amman (JO); Lina Najati Nabulsi, Amman (JO); Mahmoud M. Omari, Amman (JO)

Correspondence Address: MERCHANT & GOULD PC, P.O. BOX 2903, MINNEAPOLIS, MN, 55402-0903, US

(73) Assignee: The Jordanian pharmaceutical manufacturing Co., Naor (JO)

(21) Appl. No.: 10/796,451

(22) Filed: Mar. 9, 2004

(30) Foreign Application Priority Data
Mar. 19, 2003 (EP) 03006160.0

(51) Int. Cl. A61K 31/496; A61K 31/47; A61K 9/20

(52) U.S. Cl. 424/464; 514/253.08; 514/312

(57) ABSTRACT

A wet granulation formulation for oral dosage forms containing a non-hydrated quinoline carboxylic acid type antibacterial agent with low water content and non-hygroscopic properties.

### BACKGROUND OF THE INVENTION

The invention relates to the formulation of non-hydrated quinoline carboxylic acids with various additives to produce non-hygroscopic formulas for oral dosage forms.

Said compounds are mainly hygroscopic and can form different hydrates. For example, norfloxacin in its anhydrous form suffers from water uptake in its pure state or in solid dosage forms. It can absorb water to produce hydrates with different molar ratios (sesquihydrate, dihydrate). The water percentages are 7.5% and 10% for sesquihydrate and dihydrate, respectively.

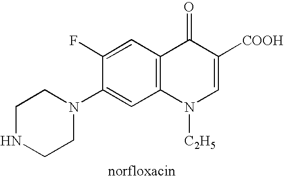

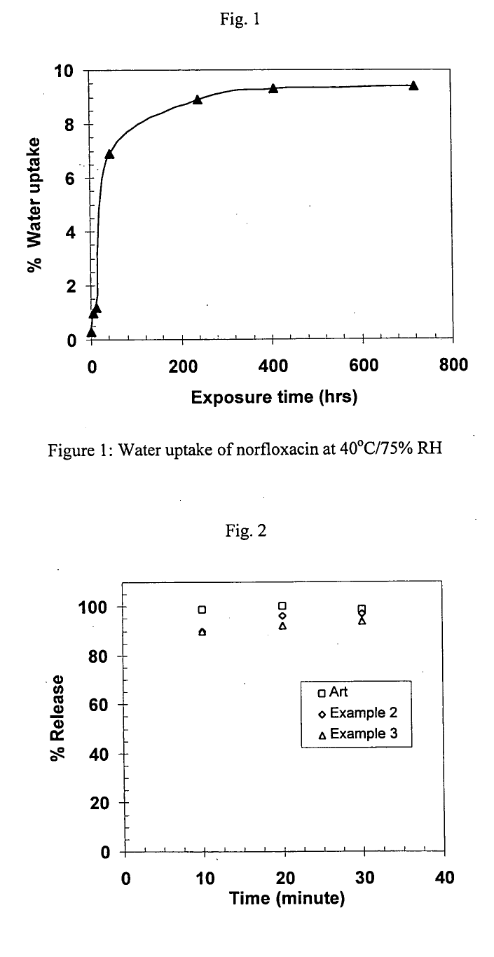

The water uptake for norfloxacin and its formulations may reach up to 30% and more depending on the manufacturing procedures (wet or dry granulation), and storage conditions of temperature and humidity (see FIG. 1).

In Italian patent application 20764A/79, water (about 2% to about 15%) is included in formulations for tablet oral dosage forms. From the manufacturing process and control point of views it is not favorable to have a significant variation in water content. Also, formulation of a hygroscopic compound such as norfloxacin with low water content is liable to accommodate more water. These types of formulations may require a high protective package such as aluminum/aluminum.

A direct compression tablets formulation for quinoline carboxylic acids with low water content (<2%) is disclosed in European patent application EP 189 114. In ths patent application, the formulation contains an antibacterial agent, preferably norfloxacin, and minimal amounts of disintegrant, filler/binder and lubricant. Such formulations require a high protective package such as aluminum/aluminum. In addition, direct compression may be considered as a critical manufacturing process, especially for formulations containing high amounts of the active ingredient such as norfloxacin (e.g. 400 mg per tablet). As a result, control of physical properties of the active ingredient such as the particle size distribution is required.

### SUMMARY OF THE INVENTION

In this invention, different formulas of non-hydrated quinoline carboxylic acids are disclosed produced by using wet granulation with water. The obtained formulas have low water content (<3%) and are non-hygroscopic.

A wet granulation tablet formulation has been discovered where water is included in a granulation step, followed by drying to obtain granules of low water content (<3%) and being non-hygroscopic compared with prior art formulations, while maintaining equivalent characteristics (dissolution, disintegration, bioavailability and physical properties) of the tablet prepared therefrom. The wet granulation formulation may be applied for other quinoline carboxylic acids.

### DESCRIPTION OF THE INVENTION

This invention covers a wet granulation formulation of quinoline carboxylic acid antibacterial agents.

Norfloxacin, ciprofloxacin, ofloxacin, enrofloxacin, lemofloxacin, levofloxacin, enoxacin, pefloxacin, balofloxacin, clinafloxacin, difloxacin, fleroxacin, grepafloxacin, gatifloxacin and the like are examples of useful quinoline carboxylic acids. A preferred quinoline carboxylic acid is norfloxacin.

The present formulation involves granulating of said antibacterial agent with a stabilizer, preferably in the presence of processing aids. The processing aids may include a filler, a disintegrant, a binder and/or a lubricant.

The stabilizer is selected from inorganic acids (e.g. hydrochloric, sulfuric or phosphoric acid) or organic acids (e.g. anhydrous citric, hydrate citric, fumaric, malic, maleic, tartaric, glutaric, benzenesulfonic, benzoic or salicylic acid).

The amount of stabilizer is 10-35% wt/wt, more favorable 20-35% wt/wt and the optimal is 35% wt/wt.

The present wet formulation includes granulation of an antibacterial agent with the stabilizer dissolved in water, ethanol or a mixture of water/ethanol (10/90 to 90/10 v/v). The obtained granules have a water content less than 2-3% after drying at a temperature range of 55° C.-65° C. Then a filler, a disintegrant and a lubricant are added followed by compression to produce tablet. The tablet is film coated using a known aqueous coating system. A modified cellulose e.g. hydroxypropylcellulose and/or hydroxypropylmethylcellulose may, for example, be used as film former.

The following examples illustrate tablet formulations containing 400 mg of an antibacterial agent (norfloxacin).

### EXAMPLE 1

### Comparative Example

| No | Component | Function | Mg/tablet | Weight % |
|---|---|---|---|---|
| 1 | Norfloxacin | Active ingredient | 400 | 80 |
| 2 | Microcrystalline | Filler | 85.5 | 17 |
| | cellulose | | | |
| 3 | Croscarmellose sodium | Disintegrant | 10 | 2 |
| 4 | Magnesium stearate | Lubricant | 4.5 | 1 |

### EXAMPLE 2

### Formulation of Invention

| No | Component | Function | mg/tablet | Weight % |
|---|---|---|---|---|
| 1 | Norfloxacin | Active ingredient | 400 | 63.5 |
| 2 | Anhydrous citric acid | Stabilizer | 200 | 31.7 |
| 3 | Sodium starch glycolate | Disintegrant | 23 | 3.7 |
| 4 | Magnesium stearate | Lubricant | 7 | 1.1 |

The tablet preparation is carried out by granulating the antibacterial agent (norfloxacin) with water in the absence of stabilizer (example 1) and in the presence of stabilizer (examples 2 and 3). Example 3 is the same as in example 2, but a mixture of water/ethanol (50/50, v/v) is used instead of water. The amount of stabilizer represents about 50% wt/v compared to granulation solvent (i.e. 200 mg citric acid dissolved in 400 ml water). Drying at a temperature of 60° C. to obtain suitable granules is carried out. Sizing of granules is carried out followed by addition of a filler, a disintegrant and a lubricant.

The water contents of the powder obtained in example 1 and 2 are 12% and 2.2%, respectively. This indicates that the presence of stabilizer prevents the active ingredient (norfloxacin) to form hydrates. In addition, incubation of powder obtained in example 2 at 40° C. for 3 months in an open container do not show any significant increase in water content.

The tablet preparation is carried out by granulating an antibacterial agent (norfloxacin) with stabilizer dissolved in a suitable amount of water or a mixture of water/ethanol (50/50, v/v). The amounts of stabilizer represent about 10-35%. The amount of stabilizer represents about 50% wt/v compared to granulation solvent (i.e. 200 mg citric acid dissolved in 400 ml water). Both granulation solvents may be used, but a mixture of water/ethanol is more applicable. Drying to obtain suitable granules with a water content of less than 3% is carried out at a temperature range of 55° C.-65° C. Sizing of granules is carried out followed by addition of a filler, a disintegrant and a lubricant. A filler such as microcrystalline cellulose may enhance the powder flowability. The final mix is compressed to produce tablets. The tablet may be film coated using a water-based system.

Stability study of norfloxacin in the presence of stabilizer is monitored by chromatographic methods (U.S. Pat. No. 25,2002, under norfloxacin and norfloxacin tablets). The stability results at 40° C./75% RH for 3 months in open and closed containers shows no sign of instability.

The present invention produces a non-hygroscopic formulation although wet granulation in the presence of water is used. This provides process advantages such as using low water protective packaging material such as PVC, reproducible formulas with a water content of less than 3%, less sensitive as wet granulation for the variation of norfloxacin particle sizes, and low cost process.

The following table represents the physical properties of tablets containing 400 mg norfloxacin obtained from EP 189 114 and according to the present formulation.

| Tablet | Disintegration | Hardness | Dissolution¹ (%) | | |
|---|---|---|---|---|---|
| formulation | time (min) | (N) | 10 min | 20 min | 30 min |
| EP 189 114 | <5 | 164-182 | 99 | 100 | 99 |
| Example 2 | <5 | 94-105 | 90 | 96 | 97 |
| (water)* | | | | | |
| Example 3 | <5 | 124-140 | 90 | 92 | 94 |
| (50% ethanol)* | | | | | |

*Granulation solvent,
¹using USP method under Norfloxacin Tablets

The data as shown in the above table and FIG. 2 indicate that both formulations of the EP 189 114 and the present patent produce tablets of fast disintegration and dissolution profiles. Both formulations are substantially equivalent.

Tablets containing more or less than 400 mg can be prepared as needed.

The features disclosed in the foregoing description, in the claims and/or the accompanying drawings may both separately and in any combination thereof be material for realising the invention in diverse forms thereof.

1. A composition, for use and preparing a tablet by wet granulation, containing at least one quinoline carboxylic acid antibacterial agent and a stabilizer selected form the group consisting of inorganic acids such as hydrochloric, sulfuric or phosphoric acid, or the group of organic acids, such as anhydrous citric acid, hydrated citric acid, furmaric acid, malic acid, maleic acid, tartaric acid, glutaric acid, benzenesulfonic acid, benzoic acid or salicylic acid.

2. The composition of claim 1, wherein the stabilizer is anhydrous citric acid.

3. The composition of claim 1 wherein the amount of stabilizer is 10 to 35% wt/wt.

4. The composition of claim 1 wherein the amount of the stabilizer is 20 to 35% wt/wt.

5. The composition of claim 1 wherein the amount of stabilizer is around 35% wt/wt.

6. The composition of claim 1 wherein the quinoline carboxylic acid is norfloxacin.

7. The composition of claim 4 containing, by weight, about 60-70% norfloxacin, about 10-35% of a stabilizer.

8. The composition of claim 7 wherein the composition contains at least about 65% by weight of norfloxacin.

9. The composition of claim 1 wherein the composition also contains inert diluents such as filler/binder, a disintegrant and/or a lubricant.

10. The composition of claim 9 wherein the composition contains about 5-15% of a binder/filler, 1-5% of a disintegrant and/or 0.5-2% of a lubricant.

11. The composition of claim 10 wherein the stabilizer is anhydrous citric acid, the filler/binder is a microcrystalline cellulose, the disintegrant is sodium starch glycollate and the lubricant is magnesium stearate.

12. A tablet prepared from the composition of claim 1.

13. A tablet of claim 12 which has a conventional film coating.

14. A method of preparing a tablet comprising a composition according to claim 1, which composition contains at least one quinoline carboxylic acid antibacterial agent and at least one stabilizer selected from the group consisting of inorganic acids or organic acids, comprising the steps of:
(i) granulating the antibacterial agent with the stabilizer dissolved in water, ethanol or a mixture of water/ethanol (10/90 to 90/10 v/v),
(ii) drying the obtained granules to a water content less than 3%, and
(iii) compressing the granules into a tablet.

15. The method according to claim 14, wherein the granules are dried at temperature range of 55° C.-65° C.

16. The method according to claim 14, wherein the method further comprises adding processing aids after step (ii) and before compressing.

17. The method according to claim 14, wherein the compressed tablet is film coated with a film forming system.

18. The method according to claim 17, wherein the film forming system is a modified cellulose, such as hydroxypropylcellulose and/or hydroxypropylmethylcellulose.

19. A tablet prepared by the method of claim 14.
